(19) United States
(12) Patent Application Publication
(10) Pub. No.: US 2017/0296584 A1
(43) Pub. Date: Oct. 19, 2017

(54) METHODS FOR EXPANDING T CELL POPULATIONS

(71) Applicant: TEL HASHOMER MEDICAL RESEARCH INFRASTRUCTURE AND SERVICES LTD., Ramat Gan (IL)

(72) Inventors: Ilan BANK, Ramat HaSharon (IL); Michal BESSER, Tel Aviv (IL); Victoria MARCU, Givatayim (IL)

(73) Assignee: TEL HASHOMER MEDICAL RESEARCH INFRASTRUCTURE AND SERVICES LTD., Ramat Gan (IL)

(21) Appl. No.: 15/517,339

(22) PCT Filed: Sep. 24, 2015
(86) PCT No.: PCT/IL2015/050977

§ 371 Date: Apr. 6, 2017

Related U.S. Application Data
(60) Provisional application No. 62/060,027, filed on Oct. 6, 2014.

(51) Int. Cl. A61K 35/17; C12N 5/0783

(52) U.S. Cl. CPC A61K 35/17; C12N 5/0636; C12N 2501/2302; C12N 2501/999; C12N 2501/515

(57) ABSTRACT

The present invention provides methods for expanding populations of T cell. The present invention further provides pharmaceutical compositions comprising the expanded T cells, including, γδ T cells, and use thereof for treating infectious, autoimmune or malignant diseases.

### FIELD OF THE INVENTION

The present invention relates to methods for expanding populations of T cell, including γδ T cells. The present invention further relates to pharmaceutical compositions comprising the expanded T cells and uses thereof for treating infectious, malignant and autoimmune diseases.

### BACKGROUND OF THE INVENTION

Despite scientific and medical advances, cancer and infectious diseases are still a leading cause of death worldwide, and primarily in developed countries. The role of the immune system in controlling these diseases is known and its manipulation has become a highly potential treatment strategy.

Cell based immunotherapies were proved effective for some cancers. Manipulation of T cell population was found to induce disease regression and tumor cells death. For example, autologous tumor-infiltrating lymphocytes were used for treating patients with metastatic melanoma (Rosenberg et al. Science 1986, 233:1318-1321). Infusions, into cancer patients, αβ T cells recognizing antigens expressed on tumor cells, was found to induce leukemic tumor cell death resulting in clinical improvement and even recovery. However, αβ T cell therapy benefits only a small percentage of patients, indicating the need for further forms of treatments.

Recently, the potential of γδ T cells in treating cancer and infectious diseases has been recognized, however, in patients having cancer patients or chronic infections, the number and function of γ9δ2 T cells is suppressed. It has been shown that administration of autologous ex-vivo expanded γ9δ2 T cells induces anti-cancer effect, occasionally leading to cure of metastatic disease.

U.S. Pat. No. 8,609,410 discloses a method for activation of antigen-presenting cells, the method comprises co-pulsing the antigen-presenting cells in vitro with a bisphosphonate and a disease antigen.

U.S. patent application Ser. No. 13/001,581 (Publication No. 2012/0107292) discloses a method for culturing disease antigen specific cytotoxic T lymphocytes (CTLs) and γδT cells, comprising adding to the culture of peripheral blood mononuclear cells separated from the blood, aminobisphosphonate and a disease antigen and carrying out the culturing procedure until antigen specific CTLs and γδT cells proliferate and reach a number effective for treatment.

Itzhaki et al. (J Immunothe. 34(2):212-220, 2011) describe large-scale expansion of tumor infiltrating lymphocytes using anti-CD3 antibody and IL-2.

Lopez et al. (Blood, 96(12):3827-3837, 2000) discloses that γδ-T cells are exquisitely sensitive to apoptosis induced by T-cell mitogens OKT3 (anti-CD3 antibody) and IL-2.

There remains an unmet need for efficient ex vivo T cell expansion platforms, particularly γδ T cells, which can provides a significant yield of viable and potent cells that may be useful for immunotherapy.

### SUMMARY OF THE INVENTION

The present invention provides methods for large-scale activation and expansion of T cell populations, including populations of γδ T cells. The T cells may be derived from peripheral blood mononuclear cells (PBMC) of a subject in need of immunotherapy with said cells or from another subject. The expansion method comprises primarily two phases of expansion, including a first expansion phase based on zoledronic acid stimulation and, optionally, interleukin-2 (IL-2) and a second expansion phase comprising incubation with αCD3 and irradiated feeders PBMC (peripheral blood mononuclear cells, optionally, allogeneic) and IL-2.

The present invention further provides methods of treating a subject in need thereof comprising autologous or allogeneic transplantation of γδ T cells derived from PBMC of said subject or another subject, respectively, following expansion of said cells thereby enabling numerous cycles of transplantation required to achieve medical improvement or cure.

Thus, as demonstrated herein below, sequentially applying zoledronic acid stimulation with IL-2 followed by negative (or positive) selection for γδ T cell population, and subsequent incubation with αCD3 and IL-2 provides exceptionally high yield of viable and competent γδ T cells. Thus, the methods of the invention are suitable for adoptive immunotherapy, allowing an efficient and prolonged treatment.

There is provided, according to some embodiments, a method for the rapid expansion of T cells, the method comprising the steps of:
(i) providing peripheral blood mononuclear cells comprising a population of T cells;
(ii) incubating said peripheral blood mononuclear cells (PBMC) in a first culture medium comprising at least one bisphosphonate for a first time period thereby obtaining a first phase expansion of said population of T cells; and
(iii) incubating said peripheral blood mononuclear cells in a second culture medium comprising an α-CD3 antibody and IL-2 for a second time period, thereby obtaining a second phase expansion of said population of T cells, wherein said second phase expansion is at least 100-fold expansion.

It is to be understood that the fold of expansion disclosed herein refers to the fold change in the number of cells at the end of certain (first or second) expansion phase, relative to the number of cells that entered that specific phase. Expansion of the population of T cells after being subjected to steps (ii) is also termed ‘first phase expansion’, ‘phase I expansion’ or ‘PIE’, where expansion of the population of T cells after being subjected to steps (iii) is also termed ‘second phase expansion’, ‘rapid expansion procedure’ or ‘REP’.

In some embodiments, the population of T cells comprises γδ T cells. In some embodiments the population of T cells is consisting of γδ T cells.

In some embodiments, the peripheral blood mononuclear cells are obtained from a subject afflicted with malignant, autoimmune or infectious disease. In some embodiments the peripheral blood mononuclear cells are obtained from a healthy afflicted with malignant, infectious or autoimmune diseases.

In some embodiments, said first phase expansion is at least 100-fold, at least 200-fold, at least 300-fold, at least 400-fold or at least 500-fold expansion of said population of T cells, relative to the initial number of cells in this population prior to the first phase expansion.

In some embodiments, said first phase expansion is within the range of 100 to 1000 fold expansion of said population of T cells, relative to the initial number of cells in this population prior to the first phase expansion.

In some embodiments, said population of T cells is consisting of γδ T cells and said first phase expansion is within the range of 100 to 1000 fold expansion of said population of γδ T cells, relative to the initial number of cells in this population prior to the first phase expansion, i.e. relative to the number of γδ T cells in the initial PBMC provided in step (i).

In some embodiments, said second phase expansion is at least 100-fold, at least 200-fold, at least 300-fold, at least 400-fold or at least 500-fold expansion of said population of T cells, relative to the number of cells in this population after the first phase expansion and prior to the second phase expansion.

In some embodiments, said second phase expansion is within the range of 100 to 1000 fold expansion of said population of T cells, relative to the number of cells in this population after the first phase expansion and prior to entering the second phase expansion.

In some embodiments, said population of T cells is consisting of γδ T cells and said second phase expansion is within the range of 100 fold to 1000 fold, or 500 fold to 1000 fold, expansion of said population of γδ T cells, relative to the number of cells in this population after the first phase expansion and prior to the second phase expansion.

In some embodiments, said population of T cells is consisting of γδ T cells, wherein said γδ T cells undergo an overall expansion within the range of 10⁴-fold to 10⁶-fold following said first phase expansion and said second phase expansion. IN some embodiments, the method further comprises transplanting the population of T cells obtained in step (iii) or a fraction thereof to said subject.

In some embodiments, the method further comprises selecting and isolating T cells from said peripheral blood mononuclear cells prior to step (iii) and incubating the isolated T cells in said second culture medium. In some embodiments, the selecting comprises negative selection. In some embodiments, the selecting comprises positive selection.

In some embodiments, the method further comprises selecting and isolating γδ T cells following step (iii).

In some embodiments, the method further comprises an additional selection step comprising a negative selection using immunomagnetic columns, following any one of steps (ii) or (iii).

In some embodiments, the first culture medium further comprises IL-2.

In some embodiments the peripheral blood mononuclear cells in step (i) are having a cell density of about 0.5×10⁶ to 1×10⁶ cells/ml.

In some embodiments, the second time period is at least 10 days.

In some embodiments, the at least one bisphosphonate is selected from the group consisting of zoledronic acid, pamidronic acid, alendronic acid, risedronic acid, ibandronic acid, incadronic acid, etidronic acid, risedronic acid, tiludronic acid, a combination thereof, a salt thereof and a hydrate thereof. Each possibility is a separate embodiment of the invention.

In some embodiments the at least one bisphosphonate is zoledronic acid.

In some embodiments, the second culture medium further comprises irradiated peripheral blood mononuclear cells.

There is provided, according to some embodiments, a pharmaceutical composition comprising a population of T cells obtained by:
(i) providing peripheral blood mononuclear cells comprising a population of T cells;
(ii) incubating said peripheral blood mononuclear cells in a first culture medium comprising at least one bisphosphonate for a first time period, thereby obtaining a first phase expansion of said population of T cells; and
(iii) incubating said peripheral blood mononuclear cells retrieved from the first culture in a second culture medium comprising an α-CD3 antibody and IL-2 for a second time period, thereby obtaining a second phase expansion of said population of T cells, wherein said second phase expansion is at least 100-fold expansion.

In some embodiments, the pharmaceutical composition further comprises at least one pharmaceutically acceptable carrier or diluent.

In some embodiments, the pharmaceutical composition comprises a therapeutically effective amount of T cells.

In some embodiments, the population of T cells comprises γδ T cells derived from PBMC. In some embodiments, the population of T cells is consisting of γ9δ2 T cells derived from PBMC. In some embodiments, the γδ T cells comprise allogeneic γδ T cells derived from PBMC. In some embodiments, the γδ T cells comprise autologous γδ T cells derived from PBMC. In some embodiments, the pharmaceutical composition comprises autologous γδ T cells derived from PBMC of a subject in need of γδ T cells transplantation. In some embodiments, the pharmaceutical composition comprises allogeneic γδ T cells derived from PBMC of a subject in need of γδ T cells transplantation.

In some embodiments, the pharmaceutical composition comprises autologous γδ T cells obtained from autologous peripheral blood mononuclear cells following incubation of said peripheral blood mononuclear cells in said first and said second culture media.

In some embodiments, the pharmaceutical composition comprises allogeneic γδ T cells obtained from allogeneic peripheral blood mononuclear cells following incubation of said peripheral blood mononuclear cells in said first and said second culture media.

In some embodiments, the pharmaceutical composition is in the form of cell suspension.

In some embodiments, the pharmaceutical composition is used for the treatment of an infectious, autoimmune or malignant disease in a subject in need thereof.

There is provided, according to some embodiments, a method of treating an infectious, autoimmune or malignant disease comprising:
(i) obtaining from a first subject peripheral blood mononuclear cells comprising a population of T cells;
(ii) incubating the peripheral blood mononuclear cells in a first culture medium comprising at least one bisphosphonate for a first time period, thereby obtaining a first phase expansion of said population of T cells;
(iii) incubating said peripheral blood mononuclear cells in a second culture medium comprising an α-CD3 antibody and IL-2 for a second time period, thereby obtaining a second phase expansion of said population of T cells, wherein said second phase expansion is at least 100-fold expansion; and
(iv) administering to a second subject said cells or a fraction thereof.

In some embodiments, said second subject and said first subject is the same subject. In some embodiments, said second subject is other than said first subject. In some embodiments the method further comprising selecting and isolating T cells from said peripheral blood mononuclear cells prior to step (iii) and incubating the isolated T cells in said second culture medium.

In some embodiments, the population of T cells comprises γδ T cells. In some embodiments, the T population of cells consists of γδ T cells.

In some embodiments, the method further comprises repeating step (iv) at least one more time.

In some embodiments, the method further comprises repeating step (iv) until treatment of said infectious, autoimmune or malignant disease or disorder is completed.

In some embodiments, treating said infectious or malignant disease or disorder comprises inhibiting said infectious, autoimmune or malignant disease or disorder, attenuating said infectious, autoimmune or malignant disease or disorder, achieving relief from symptoms associated with said infectious, autoimmune or malignant disease and a combination thereof.

In some embodiments there is provided a kit for expansion of T cells, the kit comprises:
(i) at least one first container comprising a first culture medium comprising at least one bisphosphonate;
(ii) at least one second container comprising a second culture medium comprising an α-CD3 antibody and IL-2; and
(iii) written instructions for use of said kit for expanding a population of T cells.

In some embodiments, the kit further comprises apparatus for selection of T cells. In some embodiments, the kit further comprises one or more columns for T cells selection. In some embodiments, the population of T cells comprises γδ T cells. In some embodiments, the population of T cells is consisting of γδ T cells.

In some embodiments, the at least one container further comprises IL-2.

In some embodiments there is provided a kit for treating an infectious, malignant or autoimmune disease, the kit comprises:
(i) at least one first container comprising a population of T cells enriched for γδ T cells in conditions affording long term storage, wherein said population of T cells is obtained by:
   a. providing peripheral blood mononuclear cells comprising a population of T cells;
   b. incubating said peripheral blood mononuclear cells in a first culture medium comprising at least one bisphosphonate for a first time period, thereby obtaining a first phase expansion of said population of T cells enriched for γδ T cells; and
   c. incubating said peripheral blood mononuclear cells in a second culture medium comprising an α-CD3 antibody and IL-2 for a second time period, thereby obtaining a second phase expansion of said population of T cells of at least 100-fold expansion enriched for γδ T cells; and
(ii) written instructions for use of said kit for transplanting said population of T cells in a subject in need thereof.

In some embodiments, the population of T cells in the at least one first container comprises a therapeutically effective amount of γδ T cells.

Further embodiments, features, advantages and the full scope of applicability of the present invention will become apparent from the detailed description and drawings given hereinafter. However, it should be understood that the detailed description, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

Exemplary embodiments are illustrated in referenced figures. It is intended that the embodiments and figures disclosed herein are to be considered illustrative rather than restrictive. The figures are listed below.

FIG. 1A shows the total number of cells during the first phase of expansion (PIE) in healthy donors (HD, n=6) and cancer patients (CP, n=2).

FIG. 1B shows number of CD3+γ9+ T cells (60-99% purity) inoculated (day 0) and following REP (day 14) in HD (healthy donors) and CP (cancer patients).

FIG. 1C shows the fold expansion of γδ T cells following REP.

FIG. 2A is a flow cytometric dot plot of PBMC of a healthy donor stained with monoclonal antibody to CD3 (Y axis) and γ9 (X axis) indicating that 2.64% of the cells are CD3+γ9+ T cells.

FIG. 2B is a flow cytometric dot plot of PBMC of a healthy donor after 14 days culture with zoledronate and IL-2, stained with monoclonal antibody to CD3 (Y axis) and γ9 (X axis) indicating that 79.6% of the cells are CD3+γ9+ T cells.

FIG. 3A is a flow cytometric dot plot of the cells in FIG. 2B after being depleted of CD4 and CD8 cells (by immunomagnetic columns) resulting with a population of cells containing 97.7% CD3+γ9+ T cells.

FIG. 3B is a flow cytometric dot plot of CD4 and CD8 cell populations within the population of cells presented in FIG. 3A (following depletion of CD4 and CD8).

FIG. 4 is a flow cytometric dot plot of the population of the cells presented in FIG. 3A, following culture with anti-CD3 antibody and IL-2, resulting with 87.1% of CD3+γ9+ T cells.

FIG. 5 shows IFNγ secretion by glioblastoma multiforme (GBM) cell lines cultured under REP with Zoledronate (Zol) or without (Med). P values were calculated for Zoledronate compared to control (Med).

FIG. 6A shows the specific cytotoxicity (%) of T98G glioblastoma multiforme cells incubated with effector γδT cells cultured with zoledronate (circle); effector αβT cells cultured with zoledronate (dashed line, diamond); effector γδT cells cultured in the absence of zoledronate (squares); and effector αβT cultured in the absence of zoledronate (triangles).

FIG. 6B shows the specific cytotoxicity (%) of U251 glioblastoma multiforme cells incubated with effector γδT cells cultured with zoledronate (circle); effector αβT cells cultured with zoledronate (dashed line, diamond); effector γδT cells cultured in the absence of zoledronate (squares); and effector αβT cultured in the absence of zoledronate (triangles).

FIG. 6C shows the specific cytotoxicity (%) of U87 glioblastoma multiforme cells incubated with effector γδT cells cultured with zoledronate (circle); effector αβT cells cultured with zoledronate (dashed line, diamond); effector γδT cells cultured in the absence of zoledronate (squares); and effector αβT cultured in the absence of zoledronate (triangles).

FIG. 6D shows the specific cytotoxicity (%) of Daudi lymphoma cells incubated with effector γδT cells cultured with zoledronate (circle); effector αβT cells cultured with zoledronate (dashed line, diamond); effector γδT cells cultured in the absence of zoledronate (squares); and effector αβT cultured in the absence of zoledronate (triangles).

### DETAILED DESCRIPTION OF THE INVENTION

The present invention provides methods for ex vivo expansion of T cells. The capability to expand viable and functioning immune cells in culture provides a potential immunotherapy tool. The teachings of the present invention further provides two steps of cells expansion, which were found to result in exceptionally high yield of a relatively rare subset of T cells, the γδ T cells. The expanded γδ T cell population may be obtained from PBMC cells of a subject and following expansion according to the methods of the invention may be used for numerous treatments through transplantation. The expanded population of γδ T cells may be sufficient for many implantations, each time a fraction of the expanded population may be reintroduced to a subject, for immunotherapy treatment.

Gamma delta T cells (γδ T cells) represent a T-cell subset population that possess a distinct T-cell receptor (TCR) on their surface. These cells are relatively rare (1-10%) among T cells in the peripheral blood. Most T cells have αβ TCR, which is composed of α and β glycoprotein chains. In contrast, γδ T cells have a TCR that is made up of one γ chain and one δ chain. Contrary to αβ T cells, activation of γδ T cells does not rely on antigen presentation by major histocompatibility complex molecules, but is instead mediated by pathogen derived antigens and self molecules that are upregulated in stresses, such as, cancer, autoimmune and infectious diseases. In adult peripheral blood, the majority of γδ T cells express TCRs composed of Vδ2 and Vγ9 gene segments.

Human γδ TCRs, especially those expressing the γ9 and δ2 genes, appear to recognize non-protein derived antigens. These antigens are common to multiple pathogenic bacteria and are also expressed in human cells at very low levels that evade recognition by circulating γ9+δ2+γδ T cells. However, these antigens (most importantly isopentenyl pyrophosphate, IPP), which are metabolites in the mevalonate pathway, become overexpressed by “sick” or “stressed” human cells, including a wide variety of cancers and bacterially and virally infected cells. Increased levels of IPP enable γ9δ2 T cells to distinguish a “stressed” cell, be it infected or cancerous, from a healthy cell. Recognition of these common antigens by the γ9δ2 TCR triggers a cytotoxic response, resulting in killing of the cancer cells and secretion of cytokines, notably IFNγ, that are critical for mounting an immune response against the cancer or infection. Another means of discriminating tumor cells from healthy cells is by the upregulation of self-antigen like heat shock proteins (HSP), and ligands of the NKG2D receptor.

Although γ9δ2 T cells are inherently present in the human body and can potentially kill cancerous cells, they appear to be insufficient to control already developed and/or spreading of cancer. Moreover, in cancer patients and in patients with chronic infections including tuberculosis and AIDS, the number and function of these cells may be suppressed. Fortunately, however, γ9δ2 T cells collected from the blood of these sick individuals, may be activated and significantly expanded in the laboratory, according to the methods of the present invention. In addition, γ9δ2 T cells collected from the blood of a healthy donor, may be activated and significantly expanded in the laboratory, according to the methods of the present invention.

The terms “healthy subject”, “healthy donor” and “healthy individual” as used herein refer to an individual who donates peripheral blood cells comprising a population of T cells, for expansion of γ9δ2 T cells according to the method of the invention, and for use in allogenic transplantation. The healthy donor may, or may not, be afflicted with a disease or disorder requiring transplantation of γ9δ2 T cells. In this context, the subject in need of γ9δ2 T cells transplantation is the acceptor. In the case of autologous transplantation, the donor and the acceptor is the same individual.

γδ T cells are sensitive in culture, showing an inclination to apoptosis upon incubation with αCD3 and IL-2 (Lopez et al., ibid). Apoptosis usually begins after 10-14 days in culture. Unexpectedly, the methods of the present invention enable two phases of expansion, which results in high yield of γδ T cells. Thus, the methods of the invention overcome the apoptosis-induced expansion hurdle.

Advantageously, the γδ T cells population obtained by the method of the invention, may be used for numerous cycles of immunotherapy treatments.

The terms “γδ T cells” and “γ9δ2 T cells” as used herein are interchangeable. Without being bound by any theory or mechanism, the combination of bisphosphonate and, optionally, IL-2 stimulation followed by culture in the presence of α-CD3 and IL-2 and, optionally, irradiated peripheral blood mononuclear cells, also termed hereinafter “feeder cells”, enable a significant expansion while cells' viability and potency are maintained.

In some embodiments, the present invention provides a method for the rapid expansion of T cells, the method comprises the steps of:
(i) providing peripheral blood mononuclear cells comprising a population of T cells;
(ii) incubating said peripheral blood mononuclear cells in a first culture medium comprising at least one bisphosphonate and IL-2 for a first time period; and
(iii) incubating said peripheral blood mononuclear cells in a second culture medium comprising an α-CD3 antibody and IL-2 and allogeneic irradiated mononuclear cells for a second time period, thereby obtaining at least 100-fold expansion of said population of T cells.

The term “cell expansion” as used herein refers to a process of expanding a population of cells resulting in a larger number of cells, relative to the number of cells in the initial population. Typically, and as taught herein, cell expansion is carried out ex vivo, for example, in petri dishes. Cells are removed from a tissue or from the peripheral blood system and are exposed to proliferative agents that amplify the population of the cells. Cell expansion may also refer to manipulations that result in faster increase in the number of cells when comparing to the normal proliferation rate. In some embodiments, the expansion rate of an expansion phase according to the invention is at least 100 fold per two weeks. In other embodiments, the expansion rate of an expansion phase according to the invention is at least 300 fold per two weeks. Cell expansion may be interchangeable with “enrichment”, referring to the enrichment of a T cell population for γδ T cells. Enrichment for γδ T cells, according to the method disclosed herein, may be at least 30%, at least 40%, at least 50%, at least 60%, at least 70%, at least 80% and at least 90% enrichment. Each possibility represents a separate embodiment of the invention.

PBMC are a good source for obtaining immune cells such as lymphocytes. In some embodiment, PBMC are derived from the blood of a subject afflicted with cancer, autoimmune or infectious disease. In some embodiment, PBMC are derived from the blood of a healthy subject. Cells of interest may be further isolated and amplified ex vivo, and later reintroduced to the patient system, serving as immunotherapy agent.

The terms “reintroduced”, “transplanted”, “implanted” and “infused” are interchangeable and refer to the administration of the expanded T cells of the invention into a subject afflicted with diseases or disorder requiring treatment using said cells. Thus, the administration includes also implantation of the expanded cells according to the invention. In some embodiments, implantation is autologous implantation.

In some embodiments, the initial PBMC isolation procedure may include the use of ficoll, a highly branched hydrophilic polysaccharide that separates layers of blood. Ficoll may be used to separate PBMC from plasma, polymorphonuclear cells and erythrocytes.

In some embodiments, the first step of expansion comprises use of one or more bisphosphonates. Bisphosphonates are drugs that are commonly used for preventing loss of bone mass. These agents are further used for treating diseases associate with bone loss and/or deformation, such as, osteoporosis, osteitis deformans, bone metastasis (with or without hypercalcaemia) and multiple myeloma.

In some embodiments, the bisphosphonate is selected from the group consisting of zoledronic acid, pamidronic acid, alendronic acid, risedronic acid, ibandronic acid, incadronic acid, etidronic acid, risedronic acid, tiludronic acid, a combination thereof, a salt thereof and a hydrate thereof. Each possibility represents a separate embodiment of the invention. In another embodiment, the bisphosphonate is zoledronic acid or zolendronate.

In some embodiments, the bisphosphonate concentration is from 0.1 to 10 μM. In some embodiments, the bisphosphonate concentration is from 0.5 to 6 μM. In some embodiments, the bisphosphonate concentration is from 1 to 3 μM. In some embodiments, the bisphosphonate concentration is about 2 μM.

The term “about” as used herein means approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, optionally, 10 percent, up or down (higher or lower).

According to some embodiments, additional proliferative agents may be added to the first expansion step. In some embodiments, interleukin 2 (IL-2) is added to the first culture medium. In some embodiments, IL-2 is recombinant human IL-2. In some embodiments, fresh medium with IL-2 is replaced every 3 days. In some embodiments, the concentration of IL-2 is from 20 to 400 international units (IU)/ml. In some embodiments, the IL-2 concentration is from 50 to 200 international units (IU)/ml. In some embodiments, the IL-2 concentration is about 100 international units (IU)/ml.

The time of the first step of expansion, also termed ‘the first time period’ is about two weeks. In some embodiments, the first time is from 7 to 20 days. In some embodiments, the first time period is from 12 to 16 days. In some embodiments, the first time period is about 14 days.

In some embodiments, the second time period is at least 7 days, at least 10 days, or about 14 days.

In some embodiments, the second time period comprises incubation of PMBC cells comprising γδ T cells with anti CD3 (α-CD3) antibody and IL-2.

In some embodiments, the second time period further comprises incubating said cells with irradiated PBMC cells.

In some embodiments the α-CD3 antibody is a monoclonal antibody. In some embodiments, the α-CD3 antibody is OKT3.

An anti CD3 includes OKT3, a monoclonal antibody targeted to the CD3 receptor on the surface of T cells. OKT3 was approved by the FDA for reducing acute rejection in patients following organ transplants.

In some embodiments, the second culture medium comprises α-CD3 antibody in a concentration from about 0.05 ng/ml to 1 μg/ml. In some embodiments, the α-CD3 antibody concentration in said second culture medium is from about 0.1 ng/ml to 0.5 μg/ml. In some embodiments, the α-CD3 antibody concentration is from about 2 ng/ml to 100 ng/ml. In some embodiments, the α-CD3 antibody concentration in said second culture medium is from about 20 ng/ml to 40 ng/ml. In some embodiments, the α-CD3 antibody concentration in said second culture medium is about 30 ng/ml.

Following the first and/or second step of expansion, the cells may further undergo a selection step. The selection step is intended to improve the specificity of the expansion method, namely, focus the method to expansion of specific cells. In some embodiments, the selection step is a negative selection using immunomagnetic columns. In some embodiments, the selection step is a positive selection. For example, positive selection may include staining the PBMC with a murine monoclonal antibody to the Vγ9 epitope, and passaging the cells on immunomagnetic anti murine columns Cells bound to the column are the enriched cells that are required for further processing.

In some embodiments, the method of the invention further comprises selecting and isolating T cells from peripheral blood mononuclear cells prior to step (iii) and incubating the isolated T cells in said second culture medium.

In some embodiments, the method of the invention further comprises selecting and isolating γδ T cells from peripheral blood mononuclear cells prior to step (iii) and incubating the isolated γδ T cells in said second culture medium.

In some embodiments, γδ T cells are selected and isolated (purified) using immune-depletion of CD4 and CD8 cells applying specific monoclonal antibodies (mAb). Following the binding to mAb, cells are bounded to IgG micro-beads and passed through MACS columns. In some embodiments, the selection is for γδ T cells using anti γδ TCR monoclonal antibodies.

The cells that undergo expansion according to the invention may be used in immunotherapy for treating cancer, autoimmune and infectious diseases, via autologous or allogenic transplantation.

In some embodiments, the method further comprises selecting and isolating γδ T cells following step (iii).

In some embodiments, the first culture medium further comprises interleukin-2.

In some embodiments, peripheral blood mononuclear cells are provided at a cell density of about 0.5×10⁶ to 1×10⁶ cells/ml.

In some embodiments, the second culture medium further comprises irradiated peripheral blood mononuclear cells.

The method of the invention can be used as immunotherapy for treating malignant, autoimmune or infectious disease or autoimmune diseases. Human γδ T cells were found to kill a vast repertoire of tumor cells in vitro, including leukemia, lymphoma, melanoma, neuroblastoma, and multiple types of carcinoma.

The term “malignant” is used herein in its broadest sense and refers to a family of diseases characterized by uncontrolled cell growth. It includes, but is not limited to, adrenocortical carcinoma, anal cancer, bladder cancer, ependymoma, medulloblastoma, supratentorial primitive neuroectodermal, pineal tumors, hypothalamic glioma, breast cancer, carcinoid tumor, carcinoma, cervical cancer, colon cancer, endometrial cancer, esophageal cancer, extrahepatic bile duct cancer, ewings family of tumors (pnet), extracranial germ cell tumor, eye cancer, intraocular melanoma, gallbladder cancer, gastric cancer, germ cell tumor, extragonadal, gestational trophoblastic tumor, head and neck cancer, hypopharyngeal cancer, islet cell carcinoma, laryngeal cancer, leukemia, acute lymphoblastic, leukemia, oral cavity cancer, liver cancer, lung cancer, small cell lymphoma, AIDS-related, lymphoma, central nervous system (primary) lymphoma, cutaneous T-cell lymphoma, Hodgkin's disease, non-Hodgkin's disease, malignant mesothelioma, melanoma, merkel cell carcinoma, metastatic squamous carcinoma, multiple myeloma, plasma cell neoplasms, mycosis fungoides, myelodysplastic syndrome, myeloproliferative disorders, nasopharyngeal cancer, neuroblastoma, oropharyngeal cancer, osteosarcoma, ovarian epithelial cancer, ovarian germ cell tumor, ovarian low malignant potential tumor, pancreatic cancer, exocrine, pancreatic cancer, paranasal sinus and nasal cavity cancer, parathyroid cancer, pheochromocytoma cancer, pituitary cancer, plasma cell neoplasm, rhabdomyosarcoma, rectal cancer, renal cell cancer, salivary gland cancer, Sezary syndrome, Kaposi's sarcoma, small intestine cancer, soft tissue sarcoma, thymoma, malignant thyroid cancer, urethral cancer, uterine cancer, sarcoma, unusual cancer of childhood, vaginal cancer, vulvar cancer or Wilms' tumor, benign conditions associated with chemotherapy treatments, such as, lupus, rheumatoid arthritis and skin diseases. Each possibility represents another embodiment of the invention.

In some embodiments, the malignant disease is selected from the group consisting of renal cell cancer, brain cancer and lung cancer. Each possibility represents another embodiment of the invention.

The term “infectious disease” as used herein is not limited and can be a result of any pathogenic agent. Infectious disease may be, for example, the result of viral infections, such as AIDS, hepatitis B and C, cellular infections, bacterial infections, parasites and fungi.

The term “autoimmune disease” as used herein refers to diseases and disorders induced by the body's immune responses being directed against its own tissues, causing prolonged inflammation and subsequent tissue destruction. Non limiting examples of autoimmune diseases and disorders include alopecia areata, diabetes Type 1, Guillain-Barre syndrome, multiple sclerosis, rheumatoid arthritis, scleroderma, polymyositis, vitiligo and systemic lupus erythematosus among others.

In some embodiments, the present invention provides a pharmaceutical composition comprising the population of T cells obtained by the method of the invention. In some embodiments, the population of T cells comprises γδ T cells derived from PBMC. In some embodiments the population of T cells is consisting of γ9δ2 T cells derived from PBMC.

In some embodiments, the pharmaceutical composition comprises γδ T cells obtained from peripheral blood mononuclear cells following incubation of said peripheral blood mononuclear cells in a first culture medium for a first time period, then in a second culture medium for a second time period, wherein said first culture medium comprises at least one bisphosphonate, and, optionally, IL-2, said second culture medium comprises an α-CD3 antibody and IL-2, and, optionally, irradiated peripheral blood mononuclear cells. In some embodiments, said peripheral blood mononuclear cells may be derived from an allogeneic source. In some embodiments, said peripheral blood mononuclear cells may be derived from an autologous source.

As used herein, a “pharmaceutical composition” refers to a preparation of a composition comprising the T cells according to the invention, suitable for administration to a patient.

In some embodiments, the pharmaceutical composition further comprises at least one pharmaceutical acceptable carrier. In some embodiments, the pharmaceutical composition may further comprise one or more stabilizers.

The present invention further provides a method of treating an infectious, autoimmune or malignant disease or disorder in a subject in need thereof comprising:
(i) obtaining from a first subject peripheral blood mononuclear cells (PBMC) comprising a population of T cells;
(ii) incubating said peripheral blood mononuclear cells in a first culture medium comprising at least one bisphosphonate and IL-2 for a first time period, thereby obtaining a stimulated cell population;
(iii) incubating said stimulated cell population in a second culture medium comprising an α-CD3 antibody and IL-2 for a second time period thereby obtaining an expanded cell population; and
(iv) administering to a second subject said expanded cell population or a fraction thereof.

In some embodiments, said first subject is a subject having an infectious, autoimmune or malignant disease or disorder, said second subject is a donor and step (iv) is an allogenic transplantation. In some embodiments, said first subject and said second subject is a subject afflicted with an infectious, autoimmune disease, or malignant disease or disorder and step (iv) is an autologous transplantation. In some embodiments, the second culture medium further comprises irradiated peripheral blood mononuclear cells. In some embodiments, the peripheral blood mononuclear cells are allogeneic peripheral blood mononuclear cells.

In some embodiments, the fraction of said expanded cell population corresponds to a therapeutically effective amount of cells.

In some embodiments, the fraction of said expanded said population is provided in the form of a pharmaceutical composition.

As used herein, the term “therapeutically effective amount” refers to an amount of a pharmaceutical composition which prevents or ameliorates at least partially, the symptoms signs of a particular disease, e.g. infectious, autoimmune or malignant disease, in a living organism to whom it is administered over some period of time.

In some embodiments, step (iv) is repeated at least one more time. In some embodiments, step (iv) is repeated a plurality of times. In some embodiments step (iv) is repeated until a desired therapeutic effect is achieved.

In some embodiments, the present invention provides a pharmaceutical composition comprising T cells obtained by the method of the invention, for use in the treatment of an infectious or autoimmune disease or malignant disease or disorder in a subject in need thereof.

The present invention further provides a kit for expansion of T cells, the kit comprises:
(i) at least one first container comprising a first culture medium comprising at least one bisphosphonate;
(ii) at least one second container comprising a second culture medium comprising an α-CD3 antibody and IL-2; and
(iii) written instructions for use of said kit for expanding a population of T cells.

In some embodiments, the kit further comprises apparatus for selection of T cells. In some embodiments, the kit further comprises one or more columns for T cells selection. In some embodiments, the population of T cells comprises γδ T cells.

In some embodiments, the kit further comprises culture apparatus for culturing cells.

In some embodiments, the kit further comprises irradiated PBMC.

In some embodiments, the first culture medium further comprises IL-2.

In some embodiments, the second culture medium further comprises irradiated PBMC.

In some embodiments, the kit further comprises at least one third container comprising peripheral blood cells comprising a population of T cells. In some embodiments, the population of T cells comprises γδ T cells. In some embodiments, the population of T cells is consisting of γδ T cells

In some embodiments, the kit further comprises instructions for use of said at least one first container. According to some embodiments, the kit further comprises instructions for use of said at least one second container. In some embodiments, the kit further comprises instructions for coordinating the administration of cells expanded using said kit to a subject in need thereof.

In some embodiments, each of said first culture medium within the at least first container and said second culture medium within said at least second container is stored under storage conditions suitable for maintaining the stability of said media and the components included therein.

Suitable storage conditions refer to conditions required to essentially retain the physical stability and/or chemical stability and/or biological activity of the media and the biologically active components within the media upon storage. In some embodiments, each of said first and second culture media is stable at room temperature (about 25° C.) or at 30° C. for at least 1 month and/or stable at about 2-8° C. for at least 1 year, or for at least 2 years. In some embodiments, each of said first and second culture media is stable following freezing (to, e.g., −70° C.) and thawing of the media. In some embodiments, the kits of the invention further comprise antibodies for selection of specific cells. In some embodiments, the kits further comprise reagents for negative selection for γδ T cells. In some embodiments, the kits further comprise reagents for positive selection for γδ T cells.

In some embodiments there is provided a kit for treating an infectious, autoimmune or malignant disease, the kit comprises:
(i) at least one first container comprising a population of T cells enriched for γδ T cells wherein said population of T cells is obtained by:
   a. providing peripheral blood mononuclear cells comprising a population of T cells;
   b. incubating said peripheral blood mononuclear cells in a first culture medium comprising at least one bisphosphonate for a first time period;
   c. incubating said peripheral blood mononuclear cells in a second culture medium comprising an α-CD3 antibody and IL-2 for a second time period, thereby obtaining at least 100-fold expansion of said population of T cells enriched for γδ T cells; and
(ii) written instructions for use of said kit for transplanting said population of T cells in a subject in need thereof.

In some embodiments, the population of T cells in the at least one first container comprises a therapeutically effective amount of γδ T cells.

In some embodiments, said at least one container is kept under conditions affording long term storage of the population of T cells disclosed therein. In some embodiments, said at least one first kit is stored under cryogenic conditions.

Typically, the at least one container is frozen and stored. This storage of cell cultures requires relatively little time and effort for their maintenance provided that the at least one first container is maintained in an ultracold (−130° C. or lower) mechanical freezer or within liquid nitrogen. Cryogenically preserved cultures usually do not undergo any detectable changes once stored below −130° C. Thus, the kit disclosed herein is suitable for ongoing long-term culture under storage. Thus, the pharmaceutical compositions and kits of the present invention may be kept under different storage conditions allowing these products to be marketed as off-the-shelf products.

In some embodiments, the kit further comprises instructions for coordinating the administration of the cells in the at least one first container to a subject in need thereof. According to some embodiments, the kit further comprises a notice in the form described by a governmental agency regulating the manufacture, use or sale of pharmaceuticals or biological products, which notice reflects approval by the agency of manufacture, use or sale for human administration.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1—Expansion of T Cells Derived from PBMC Cells

A 100-500 ml unit of peripheral blood was drawn from a consenting individual and subjected to Ficoll hypaque density centrifugation to obtain peripheral blood mononuclear cells (PBMC). The cells were subjected to the following expansion protocol for γ9δ2 T cells, which resulted in an unexpected cell expansion of about 500 fold in each phase relative to the initial amount:

(a) phase I expansion (PIE)—cells were seeded at a density of 0.5-1×10⁶ cells/ml in culture flasks in RPMI medium supplemented with 10% fetal bovine serum, 2 mM L-glutamine and penicillin—streptomycin solution (100 μg/ml). Cultures were primed with 2 μM of Zoledronate and 100 international units (IU)/ml of recombinant human IL-2 (rhIL-2) and maintained for 14 days; culture medium was replaced with fresh medium with IL-2 is every 3 days. Following the first incubation time, γδ T cells were purified from the total cell population using immuno-depletion of CD4 and CD8 cells using specific mAb, subsequent binding of anti-mouse IgG micro-beads following passing through MACS columns. The purity of eluted γ9δ2T cells was confirmed by FACS (>90%) prior to continuing to the second stage of expansion, Phase Two of Expansion (PTE) also termed rapid expansion procedure (REP).

(b) PTE was performed in GMP-like conditions which allow using the expanded cells in patients, as was described for tumor infiltrating lymphocytes previously in details (Itzhaki et al, ibid). In short, 130,000 purified γ9δ2 T cells were cultured in 20 ml of rapid expansion procedure (REP) medium containing 50% AIM-V medium, 50% CM (RPMI 1640 containing 10% human serum), 25 mmol/L HEPES pH 7.2, 100 U/mL penicillin, 100 μg/mL streptomycin, 50 μg/mL gentamycin and 5.5×10e-5 mol/L 2-mercaptoethanol, anti-CD3 antibody (Orthoclone OKT-3, 30 ng/mL), 3,000 IU/mL IL-2 and irradiated (5000 rad) allogenic feeder cells at a 200:1 feeder to γδ T cell ratio. The mixture was placed in a vertical positioned T25 flask. At day 5-70% of the REP medium was replaced with fresh medium, containing 50% AIM-V medium, 50% CM and 3,000 IU/mL IL-2. On day 7, cells from each T25 flasks were transferred into T75 flask and 20 mL AIM-V medium containing 10% human AB serum, 100 U/mL penicillin, 100 μg/mL streptomycin, 2 mmol L-glutamine and IL-2, was added. From day 9 onwards, AIM-V medium with penicillin, streptomycin, L-glutamine and IL-2 was added every 2-3 days, keeping the cell density approximately 0.5-2×10⁶ cells per ml. When necessary, cells were split into two flasks.

The fold expansion in REP of γδ T cells obtained from 6 healthy donors (which were initially expanded in PIE and selected for γδ T cells as described above) is represented in Table 1 below. The results indicate that the second phase expansion produced fold expansions above 500. Specifically, the second phase expansion yielded fold expansions within the range of 500 to 1000.

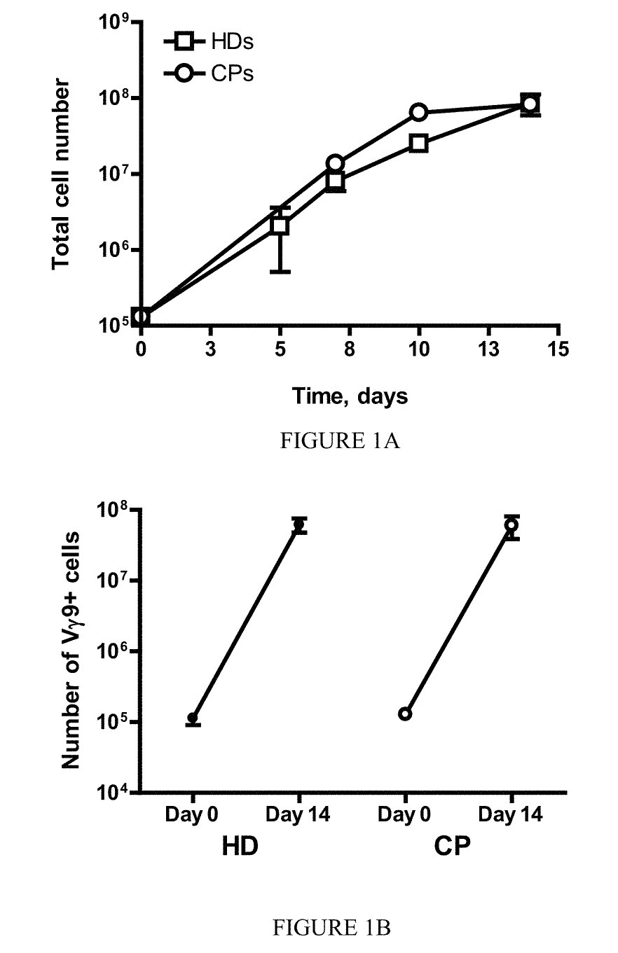

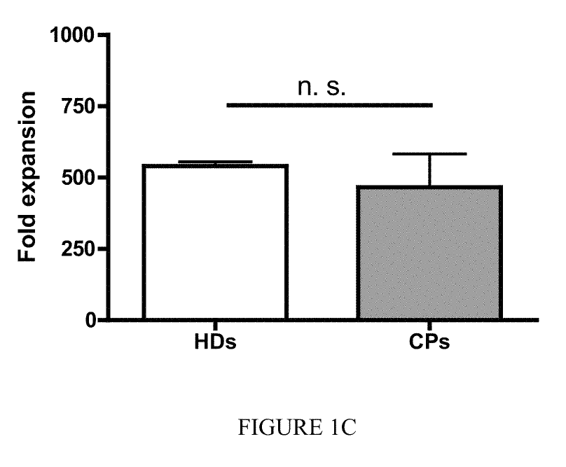

Further to Table 1, FIGS. 1A, 1B, 2A, 2B, 3A, 3B and 4 represent the aforementioned expansions. Total viable cell number was determined by microscopic cell count and trypan blue exclusion every other day, starting from day 5 (FIG. 1A). On day 14 cells were collected, counted, analyzed for γδ T cell content by flow cytometry and tested for their anti-GBM cytotoxicity. In a set of 8 PTE experiments each started with 130,000 peripheral blood cells obtained from healthy donors (HD) and patients with glioblastoma multiforme (CP; cancer patients) a yield of 8.5±1.1×10⁷ (n=6) and 8.3±0.5×10⁷ (n=2) γδ T cells was obtained for HD and CP, respectively (FIGS. 1A and 1B), corresponding to an expansion of about 600 fold (FIG. 1C). There was no significant difference (n.s) in fold expansion between the HD and CP.

TABLE 1

| | REP Phase | | | | | | |
|---|---|---|---|---|---|---|---|
| | Day 0 | | | Day 14 | | | |
| | Total | Number | | Total | Number | | |
| Healthy | Number | of γ9+ | % of γ9+ | Number | of γ9+ | % of γ9+ | γ9+ Fold |
| Donor | of Cells | T cells | T cells | of Cells | T cells | T cells | Expansion |
| HD28 | 130,000 | 127,010 | 98% | 88,000,000 | 68,640,000 | 78% | 540 |
| HD29(1) | 130,000 | 127,010 | 98% | 76,500,000 | 66,555,000 | 87% | 524 |
| HD29(2) | 130,000 | 118,560 | 91% | 133,000,000 | 63,840,000 | 48% | 538 |
| HD31 | 130,000 | 5,369 | 4% | 59,500,000 | 5,057,500 | 9% | 942 |
| HD32 | 130,000 | 73,320 | 56% | 65,000,000 | 37,050,000 | 57% | 505 |
| HD33 | 130,000 | 119,730 | 92% | 90,000,000 | 71,100,000 | 79% | 694 |
| Average | 130,000 | 95,167 | 73% | 85,333,333 | 52,040,417 | 61% | 607 |

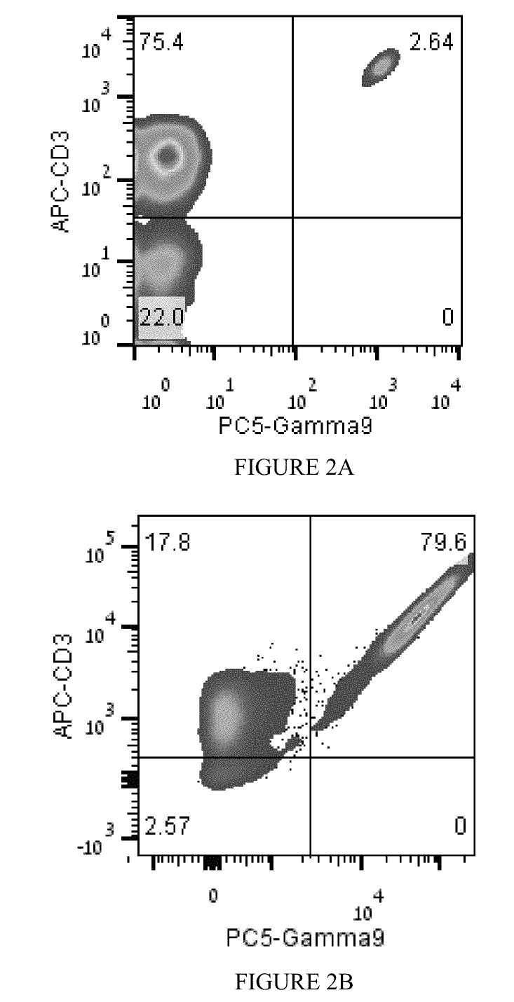

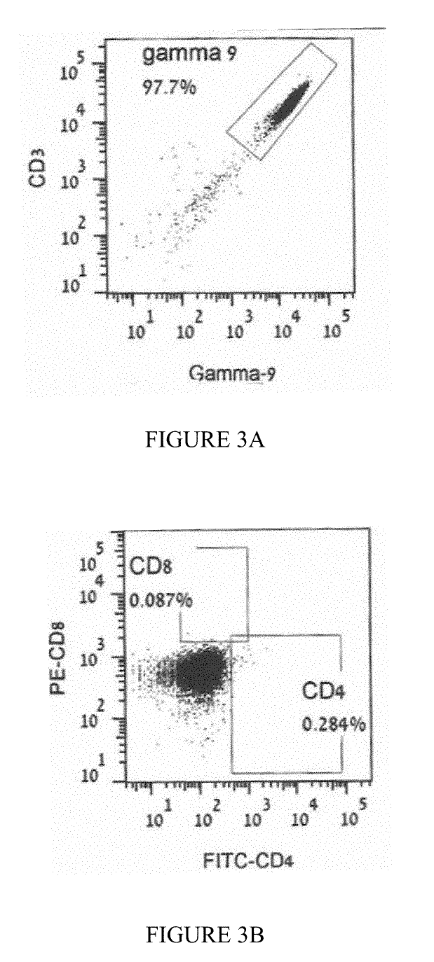

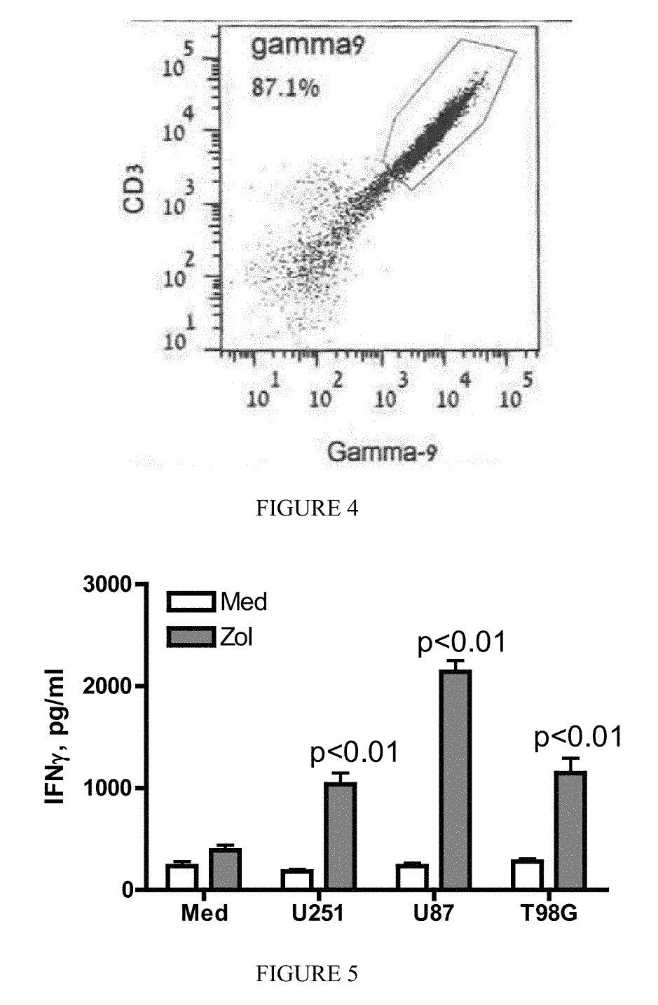

Flow cytometry analysis was performed on one exemplary sample of a healthy subject from the population studied above (FIGS. 2A, 2B, 3A, 3B and 4). Initially, a flow cytometric dot plot of PBMC stained with monoclonal antibody to CD3 (Y axis) and γ9 (X axis) was obtained (FIG. 2A). The data indicates that 2.64% of the cells are CD3+Vγ9+. Next, the cells were incubated, for 14 days, in a cell culture containing zoledronate and interleukin 2 (FIG. 2B). This first phase of expansion resulted with 79.6% cells that were stained with both antibodies, i.e. T cell expressing Vγ9 (right upper quadrant in FIG. 2B). Further enrichment for Vγ9+ T cells was achieved by passing the cells on immunomagnetic columns in order to deplete the CD4 and CD8 cells. This enrichment step resulted with 97.7% (from 79.6%) of Vγ9+ T cells. Efficiency of depletion was confirmed by the analyses shown in FIGS. 3A and 3B, where 97.7% of the cells were T cell expressing Vγ9 (right upper quadrant in FIG. 3A), only 0.08% of the cells express CD8 and 0.28% express CD4 (FIG. 3B). The analysis presented in FIG. 4 presents the percentage of CD3+Vγ9+ T cells after subjecting the cells shown in FIG. 2B to the second phase expansion, i.e. the rapid expansion protocol (REP). The results indicate that 87.1% of the T cells in this culture were CD3+Vγ9+ T cells and the fold expansion over the cells retrieved from the primary (first phase) expansion in this example was X540.

### Example 2—Activity of Expanded T Cells

The reactivity of γδT cells grown, expanded and isolated as detailed in Example 1, against GBM cell lines was tested using IFNγ secretion assay. γδ T cells were incubated with three GBM cell lines, U251, U87 and T98G, at effector to target ratio of 5:1 for 48 hours with or without Zoledronate. Following incubation, the supernatants were analyzed for IFNγ secretion. PTE derived γδT cells established certain spontaneous IFNγ secretion in the medium (Med), but IFNγ secretion in response to GBM cell lines was significantly higher (p<0.01; FIG. 5)

Overall, the results indicate that the two phase expansion method provides a high yield of viable and potent γδ T cells.

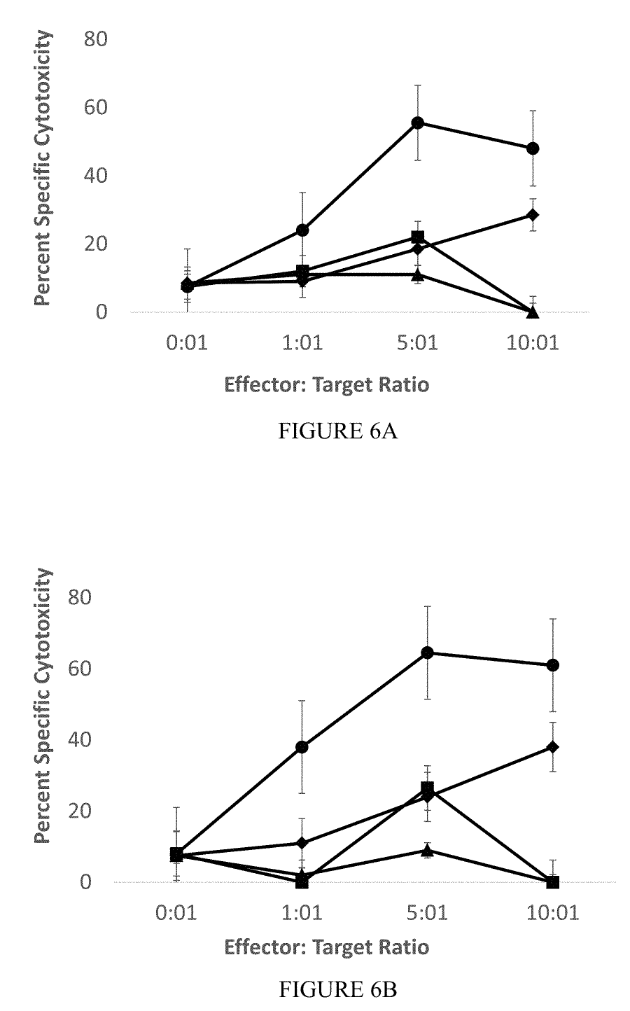

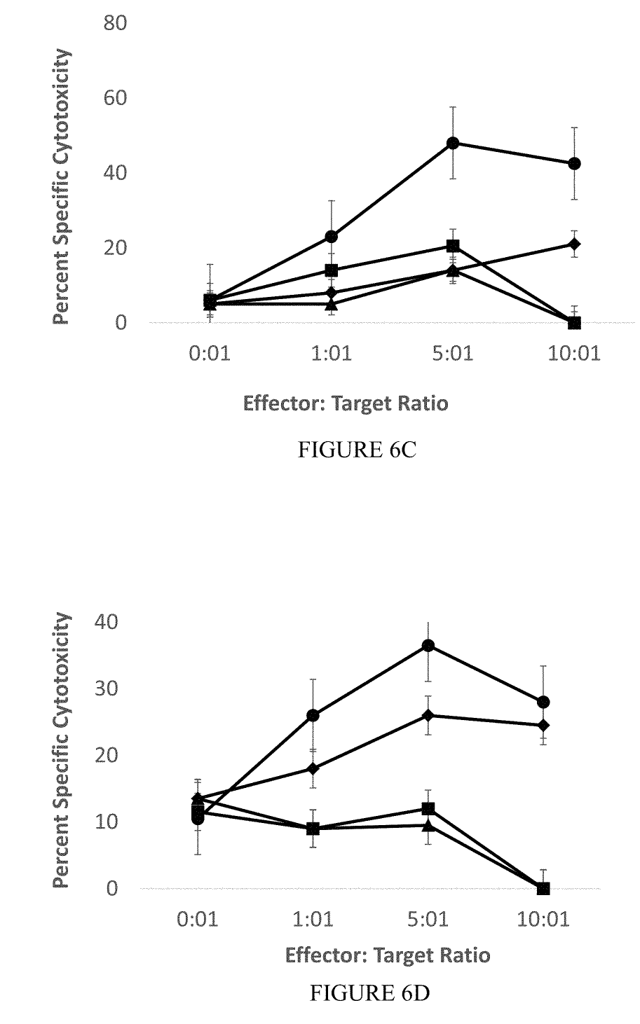

An additional study for testing the activity of the expanded effector γδT cells was conducted as follows: tumor cell lines T98G, U87, U251 (glioblastoma multiforme) and Daudi (lymphoma) target cells were labeled with 2.5 mM of 5,6-carboxyfluorescein diacetate succinimidyl ester (CF SE) (Molecular probes) for 10 min in room temperature. Thereafter CFSE was quenched with same volume of FBS (Fetal Bovine Serum) and washed with growth medium twice. Labeled target cells were plated into round bottom 96 well plate overnight together with effector γδT cells obtained by REP, or similarly activated/expanded αβ T cells (control) in various effector to target (E:T) ratios, with or without 2 mM of zoledronate. Next morning cells were harvested and stained with propidium iodide (PI). Specific lysis was measured with flow cytometry analysis of PI positive cells among CFSE labeled population. Background absorption of PI by the target cells without effectors was subtracted from the experimental samples. The results are demonstrated in FIG. 6A to 6D. The result indicate that the most significant and effective treatment (cytotoxicity) was induced by effector γδT cells cultured with zoledronate. Control cells (αβ T cells) did not exert an efficient effect compared to effector γδT cells.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-23. (canceled)

24. A method for the rapid expansion of T cells, the method comprising the steps of:
(i) providing peripheral blood mononuclear cells comprising a population of T cells;
(ii) incubating said peripheral blood mononuclear cells in a first culture medium comprising at least one bisphosphonate for a first time period, thereby obtaining a first phase expansion of said population of T cells; and
(iii) incubating said peripheral blood mononuclear cells in a second culture medium comprising an α-CD3 antibody and interleukin-2 for a second time period, thereby obtaining a second phase expansion of said population of T cells, wherein said second phase expansion is at least 100-fold expansion.

25. The method according to claim 24, wherein said population of T cells comprises γδ T cells.

26. The method according to claim 24, wherein peripheral blood mononuclear cells are obtained from a subject afflicted with malignant, autoimmune or infectious disease.

27. The method according to claim 26, further comprising transplanting the population of T cells obtained in step (iii) or a fraction thereof to said subject.

28. The method according to claim 24, further comprising selecting and isolating T cells from said peripheral blood mononuclear cells prior to step (iii) and incubating the isolated T cells in said second culture medium.

29. The method according to claim 24, further comprising selecting and isolating γδ T cells following step (iii).

30. The method according to claim 24, wherein said first culture medium further comprises interleukin-2.

31. The method of claim 24, wherein said peripheral blood mononuclear cells in step (i) are having a cell density of about 0.5×106 to 1×106 cells/ml.

32. The method according to claim 24, wherein the second time period is at least 10 days.

33. The method of claim 24, wherein the at least one bisphosphonate is selected from the group consisting of zoledronic acid, pamidronic acid, alendronic acid, risedronic acid, ibandronic acid, incadronic acid, etidronic acid, risedronic acid, tiludronic acid, a combination thereof, a salt thereof and a hydrate thereof.

34. The method of claim 24, wherein the second culture medium further comprises irradiated peripheral blood mononuclear cells.

35. The method of claim 28, wherein said second phase expansion of said population of T cells, is at least 200-fold expansion.

36. The method of claim 24, wherein said first phase expansion of said population of T cells, is at least 100-fold expansion.

37. A pharmaceutical composition comprising the population of T cells obtained by the method according to claim 24.

38. A method of treating an infectious, autoimmune or malignant disease or disorder in a subject in need thereof comprising:
(i) obtaining from a subject peripheral blood mononuclear cells comprising a population of T cells;
(ii) incubating the peripheral blood mononuclear cells in a first culture medium comprising at least one bisphosphonate for a first time period, thereby obtaining a first phase expansion of said population of T cells;
(iii) incubating said peripheral blood mononuclear cells in a second culture medium comprising an α-CD3 antibody and IL-2 for a second time period, thereby obtaining a second phase expansion of said population of T cells, wherein said second phase expansion is at least 100-fold expansion; and
(iv) administering to said subject said cells or a fraction thereof.

39. The method according to claim 38, further comprising selecting and isolating T cells from said peripheral blood mononuclear cells prior to step (iii) and incubating the isolated T cells in said second culture medium.

40. The method according to claim 38, wherein said population of T cells comprises γδ T cells.

41. The method according to claim 38, wherein said population of T cells is consisting of γδ T cells.

42. The method of claim 38, further comprising repeating step (iv) at least one more time.

43. The method of claim 38, wherein the at least one bisphosphonate is selected from the group consisting of zoledronic acid, pamidronic acid, alendronic acid, risedronic acid, ibandronic acid, incadronic acid, etidronic acid, risedronic acid, tiludronic acid, a combination thereof, a salt thereof and a hydrate thereof.
